(19) United States
(12) Patent Application Publication
(10) Pub. No.: US 2025/0135148 A1
(43) Pub. Date: May 1, 2025

(54) FLUIDIC VALVE

(71) Applicant: FluidIQ Inc., Winthrop, MA (US)

(72) Inventors: Duncan Stevenson, Johannesburg (ZA); Artemio Mendoza-Garcia, Katy, TX (US); Jacob Lunday, Highland Village, TX (US); Brian Walsh, Lynchburg, VA (US); Brian Froelke, St. Louis, MO (US); Christopher Jung, Newbury Park, CA (US)

(21) Appl. No.: 18/908,372

(22) Filed: Oct. 7, 2024

Related U.S. Application Data
(63) Continuation of application No. PCT/US23/17949, filed on Apr. 7, 2023.
(60) Provisional application No. 63/337,997, filed on May 3, 2022.
(60) Provisional application No. 63/328,599, filed on Apr. 7, 2022.

(51) Int. Cl. A61M 16/20

(52) U.S. Cl. CPC A61M 16/201

(57) ABSTRACT

A fluidic device for a mechanical ventilator has an inlet configured to receive fluid from a pressurized source. The device includes a device nozzle having a device nozzle axis, a device nozzle length, and a device nozzle width. The device includes at least one port having an opening to an environment external of the device. The device is configured so that ambient fluid enters the device through the port and mixes with a source fluid from the inlet to define a diluted fluid mixture. The device is further configured so that the diluted fluid mixture enters the device nozzle.

### PRIORITY

This patent application is a continuation of international patent application number PCT/US2023/017949, filed Apr. 7, 2023, entitled, “FLUIDIC VALVE,” which in turn claims priority from provisional U.S. patent application No. 63/328,599, filed Apr. 7, 2022, and provisional U.S. patent application No. 63/337,997, filed May 3, 2022, the disclosures of which are incorporated herein, in their entirety, by reference.

### FIELD OF THE INVENTION

Illustrative embodiments of the invention generally relate to fluidic valves and, more particularly, illustrative embodiments relate to mechanical ventilation using fluidic valves.

### BACKGROUND OF THE INVENTION

Mechanical ventilation can be a vital component of critical care services for patients, and when mechanical ventilation is not deployed properly or safely, it can have disastrous effects. The challenges of responding to the recent global pandemic caused by the coronavirus COVID-19 has shown a major shortage of mechanical ventilators due to an unexpected surge of patients suffering from life-threating respiratory failure. For example, in the U.S., there was an immediate and great need for effective, inexpensive and simple to use ventilators and resuscitators. Such ventilators or resuscitators may also be needed in future regional epidemic episodes or in under-resourced environments.

### SUMMARY OF VARIOUS EMBODIMENTS

In accordance with one embodiment of the invention, a fluidic device for a mechanical ventilator has an inlet configured to receive fluid from a pressurized source. The valve includes a device nozzle having a device nozzle axis, a device nozzle length, and a device nozzle width. The device includes at least one port having an opening to an environment external of the device. The device is configured so that ambient fluid enters the device through the port and mixes with a source fluid from the inlet to define a diluted fluid mixture. The device is further configured so that the diluted fluid mixture enters the device nozzle.

In some embodiments, the fluidic device may include a plurality of ports. Each of the ports may have a closure. The closure may be adjustably opened or closed. The device may configured so that closing a port biases the fluid flow through the device in a particular direction. In some embodiments fluid exiting the inlet is biased towards an outlet-side of the nozzle. In some embodiments fluid exiting the inlet is biased towards an exhaust-side of the nozzle. Furthermore, PIP and PEEP are selectively adjustable by closing and/or opening the one or more ports.

Among other things, the device may achieve a desired flow rate through the device nozzle that is higher than a flow rate through the inlet. The ports may allow for entrainment of air outside the device into the device. The entrained air may dilute air coming from a fluid source through the inlet. The ports may also act as a safety exhaust.

In some embodiments, the inlet and the device nozzle are formed as an integral piece. Alternatively, the inlet and the device nozzle may be separately formed. The inlet and the device nozzle may be connected via fluidic tubing. The nozzle ports may be integral with the inlet.

Various embodiments include a dilution nozzle that has a diameter. The diameter may narrow from a proximal end to a distal end of the dilution nozzle. The dilution nozzle may be part of a dilution connector that is separate from the device. The dilution connector may also include the dilution ports. The dilution connector may be coupled with the device using fluidic tubing. Ambient air may be used to dilute fluid from a fluid source using the one or more dilution ports. Furthermore, air may be exhausted to outside of the device using the one or more dilution ports. The diluted fluid may flow towards the patient outlet. In various embodiments, the fluidic device is configured to oscillate fluid flow between the patient outlet and the exhaust.

Among other things, the device may include a nozzle switcher configured to transition the device between a first configuration in which the device nozzle is fluidly coupled with the port, and a second configuration in which the device nozzle is fluidly coupled with the port. The nozzle switcher may transition the device by rotating or sliding. An axis of rotation of the switcher may be offset from the central axis of the device. In the first configuration, the device is fluidly coupled with a vented passage having an opening to the port. In the second configuration, the device is fluidly coupled a passage having blocking element for the port.

In various embodiments, the fluidic device may include a fluid expansion zone distal to the inlet. The fluid expansion zone tapers down to a nozzle having a nozzle axis, a nozzle length, and a nozzle width. A transition surface is part of a patient fluid pathway that leads to an outlet. The outlet is coupled, directly or indirectly, with a patient. The device also has a stepped surface that is part of a second fluid pathway leading to an exhaust. A splitter divides the first fluid pathway and the second fluid pathway. The splitter is asymmetrical relative to the nozzle axis. The inlet, the nozzle, and the outlet share a co-planar fluid path.

In accordance with another embodiment, a method provides mechanical ventilation to a patient. The method couples a fluidic device between a breathing circuit of a patient and a pressurized source. The fluidic device has an inlet configured to receive fluid from a pressurized source. The device also has a fluid expansion zone distal to the inlet. The fluid expansion zone leads to a nozzle having a nozzle axis, a nozzle length, and a nozzle width. A transition surface is part of a patient fluid pathway leading to an outlet. The outlet is configured to couple with a patient or a breathing circuit of a patient. A stepped surface is part of a second fluid pathway leading to an exhaust. A splitter divides the first fluid pathway and the second fluid pathway. The splitter is asymmetrical relative to the nozzle axis. The inlet, the nozzle, and the outlet share a co-planar fluid path. The method also provides air flow into the inlet of the device. Air flows out of the outlet towards the patient. The air flow out of the device produces a target PIP of between about 18 cmH20 and 30 cmH20 in the patient circuit. Air then flows out of the exhaust after the target PIP is reached, until a PEEP of between about 6 cmH20 and 14 cmH20 is achieved in the patient circuit. The steps of flowing air out of the outlet and out of the exhaust define a respiratory rate of between about 10 breaths per minute and about 30 breaths per minute. Some embodiments may define a respiratory rate of between about 16 breaths per minute and about 30 breaths per minute. Thus, in one or more examples, the geometry of the device may at least in part effectively provide the PIP, PEEP, and respiratory rate.

In some embodiments, the fluid expansion zone is configured to make the flow turbulent. The tidal volume delivered to the patient from the device is between about 200 ml and about 500 ml, or between about 220 ml and about 465 ml.

The transition surface leading to the outlet may have a radius of curvature. In some embodiments, the transition surface is stepped. Alternatively, or additionally, the transition surface may be flat. In some embodiments, the surface leading to the exhaust may be stepped. In some embodiments, the device may have a plurality of exhausts.

The device may be configured so that the splitter biases fluid flow towards the outlet. The fluid flow channel leading to the exhaust may be asymmetrical from the fluid flow channel leading to the outlet. The device fluid flow channels may have a rectangular cross-section.

In some embodiments, the device is configured to provide an IE ratio of between about 1.5 and 2.0. The device may receive an air input at a pressure of between about 3 psi and about 5 psi. Various embodiments may be used to control ventilation or to support patient ventilation.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows an external view of a fluidic device in accordance with illustrative embodiments of the invention.

FIG. 1B schematically shows a cross-sectional view of the device of FIG. 1A rotated 90 degrees around a longitudinal axis of the device.

FIG. 1C schematically shows a magnified view of a portion of the device identified in FIG. 1B.

FIG. 1D schematically shows a magnified view of a portion of the device identified in FIG. 1B.

FIG. 1E schematically shows the device of FIGS. 1A-1D with an example of scale in accordance with illustrative embodiments.

FIG. 1F schematically shows an internal view of the fluid flow channels of the device in accordance with illustrative embodiments.

FIG. 2 schematically shows the device in accordance with illustrative embodiments of the invention.

FIG. 3 schematically shows fluid flow through the device in accordance with illustrative embodiments of the invention.

FIG. 4A schematically shows a device in accordance with illustrative embodiments of the invention.

FIG. 4B schematically shows air flow characteristics and breath parameters achieved through an exemplary device of FIG. 4A.

FIG. 5A schematically shows a device in accordance with illustrative embodiments of the invention.

FIG. 5B schematically shows air flow characteristics and breath parameters achieved through an exemplary device of FIG. 5A.

FIG. 6 schematically shows various performance parameters achieved using the device in accordance with illustrative embodiments.

FIGS. 7A-7C schematically illustrate the influence of the device on PIP in accordance with illustrative embodiments of the invention.

FIGS. 8A-8B schematically illustrate the influence of the device on PEEP in accordance with illustrative embodiments of the invention.

FIG. 9 schematically illustrates the influence of the device on IE ratio settings in accordance with illustrative embodiments of the invention.

FIG. 10 schematically shows airway flow and airway pressure waveforms when the device is in use in accordance with illustrative embodiments of the invention.

FIGS. 11A-11C schematically show the device in accordance with illustrative embodiments of the invention.

FIGS. 12A-12C schematically show various configurations of biased air flow through the inlet of the device in accordance with illustrative embodiments of the invention.

FIG. 13 schematically shows an external view of the device in accordance with illustrative embodiments of the invention.

FIG. 14 schematically shows an alternative embodiment of the device in accordance with illustrative embodiments.

FIGS. 15A-15C schematically show a dilution connector of FIG. 14 in accordance with illustrative embodiments.

FIG. 16 schematically shows a cross-sectional view of an alternative embodiment of the device in accordance with illustrative embodiments.

FIG. 17 schematically shows a cross-sectional view of an alternative embodiment of the device in accordance with illustrative embodiments.

FIGS. 18A-18C schematically show details of a nozzle switcher in accordance with illustrative embodiments.

FIG. 19 shows a process of mechanical ventilation using the device in accordance with illustrative embodiments of the invention.

### DESCRIPTION OF ILLUSTRATIVE EMBODIMENTS

In illustrative embodiments, a fluidic device (also referred to as a fluidic valve) is configured to provide safe and precise ventilation to a patient. The fluidic valve may be positioned between a patient breathing circuit and a pressurized gas source and/or mechanical ventilation device. In some embodiments, the fluidic valve may be integrated within a ventilator, resuscitator, bag valve mask, etc. Additionally, or alternatively, the fluidic valve interfaces between a patient breathing circuit and a mechanical ventilation device (e.g., ventilator, bag valve mask). The fluidic valve is configured to provide safe and precise lung-protective ventilation performance characteristics to the patient. The valve includes a co-planar fluid flow path defined by an inlet, nozzle, and outlet. The valve geometry is configured to produce a desired PIP, PEEP, and RR for the patient. In various embodiments, the valve automatically transitions (also referred to as oscillating) the patient from inspiration to expiration. Additionally, the valve includes a dilution port configured to dilute a source of fluid that passes through the device towards the patient outlet. Details of illustrative embodiments are discussed below.

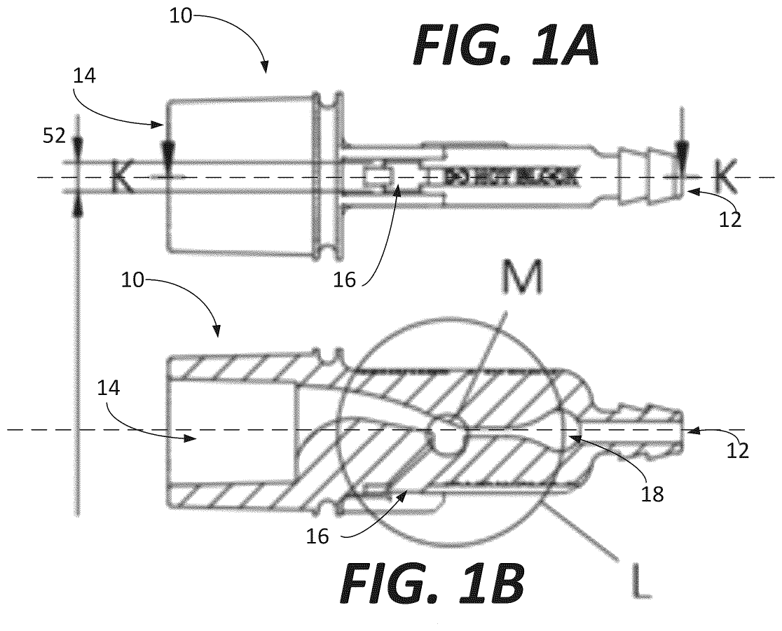

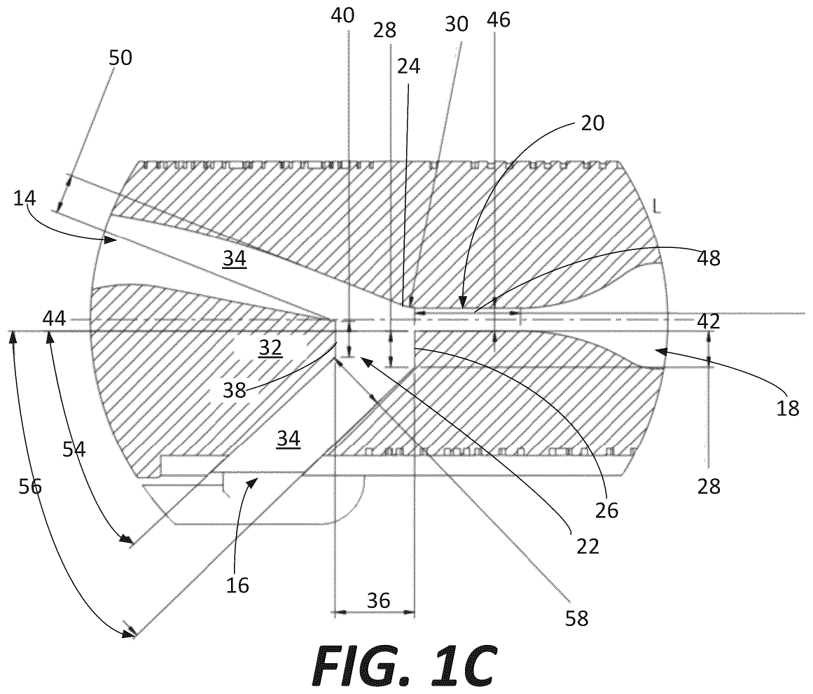

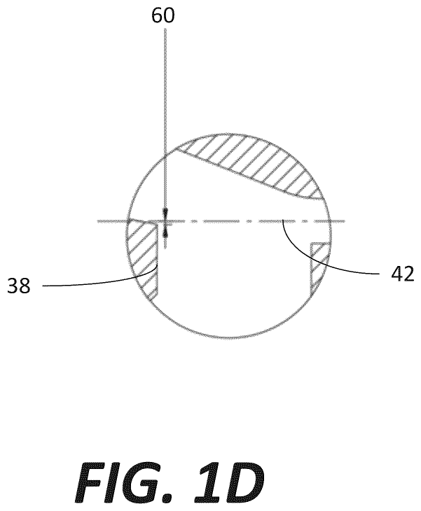

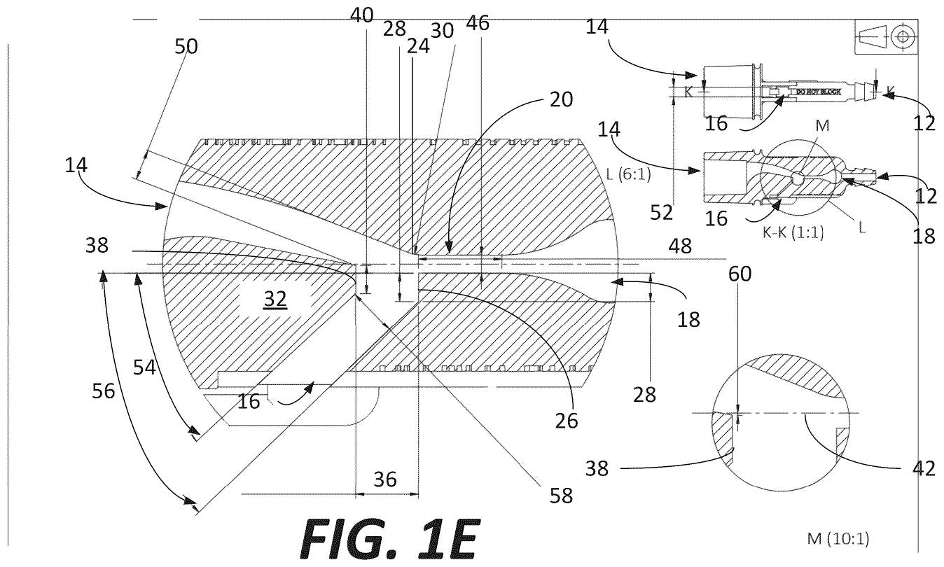

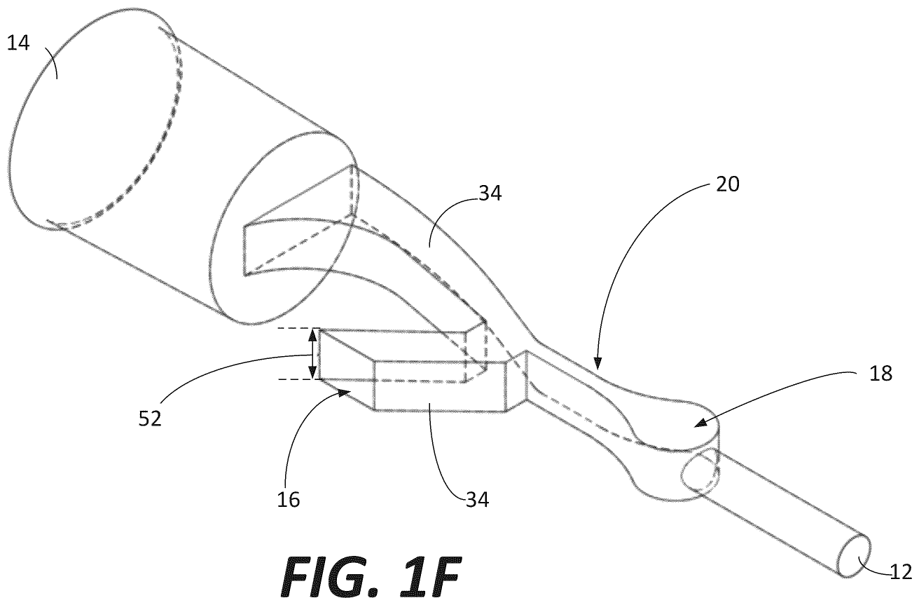

FIGS. 1A-1D schematically shows views of a fluidic valve 10 in accordance with illustrative embodiments of the invention. FIG. 1A (top right) schematically shows an external view of the fluidic valve 10 in accordance with illustrative embodiments of the invention. FIG. 1B schematically shows a cross-sectional view of the valve 10 of FIG. 1A rotated 90 degrees around a longitudinal axis of the valve 10. FIG. 1C schematically shows a magnified view of a portion L of the valve 10 identified in FIG. 1B. FIG. 1D schematically shows a magnified view of a portion M identified in FIG. 1B.

Referring to FIGS. 1A and 1B, the valve 10 has an inlet 12 configured to receive a supply of fluid (e.g., pressurized oxygen), an outlet 14 that leads to a patient, and an exhaust 16. In use, the valve 10 transitions fluid (e.g., air) flow from the outlet 14 to the exhaust 16, and vice-versa. As will be described in greater detail below, various embodiments are configured to provide precise transitioning based on desired patient parameters and also to ensure patient safety.

Referring to FIG. 1C, the fluid (i.e., air) enters through the inlet 12 into a fluid expansion zone 18 (also referred to as a power bubble 18). The fluid narrows down and passes through a nozzle 20 that leads to a split path 22 (e.g., bifurcated). The bubble 18 acts as a flow stabilizer and disrupts any boundary layer already established. In various embodiments, the nozzle 20 has a substantially uniform cross sectional area and ends at a transition surface 24. In some embodiments, the nozzle width 46 is between about 10% and 30% of the bubble 18 maximum diameter. The nozzle 20 has a nozzle width 46 and a nozzle length 48 that impacts the performance of various embodiments.

In various embodiments, the nozzle 20 terminates at a step 26 and a transition surface 24, which may be a tapered or curved surface. Alternatively, the transition surface 24 may also be a step 26 (thus both surfaces 24 and 26 after the nozzle 20 may be stepped). The step 26 has a given step offset 28, which may impact the performance of the device 10. Similarly, the transition surface 24 may have a particular radius of curvature 30 (e.g., particularly where the transition surface 24 meets the nozzle 20). The radius of curvature 30 is referred to as a the “nozzle radius 30” in various embodiments.

A splitter 32 splits the fluid pathways 34, such that the fluid may travel towards the patient outlet 14 or the exhaust 16. Various embodiments tune the splitter distance 36 (i.e., the distance from the end of the nozzle 20 to the splitter 32) to achieve desired performance characteristics. In various embodiments, the splitter distance 36 may be the distance from the step 26 to a proximal end 38 of the splitter 32. The proximal end 38 may define a splitter width 40.

The nozzle 20 defines a central nozzle axis 42, which in various embodiments, may be the same as the central longitudinal axis 44 of the valve 10. The splitter 32 may be biased to one side (e.g., the splitter 32 is not aligned with the nozzle axis 42). As shown in FIG. 1D, the proximal end 38 of the splitter 32 is misaligned from the longitudinal axis 42 of the nozzle 20.

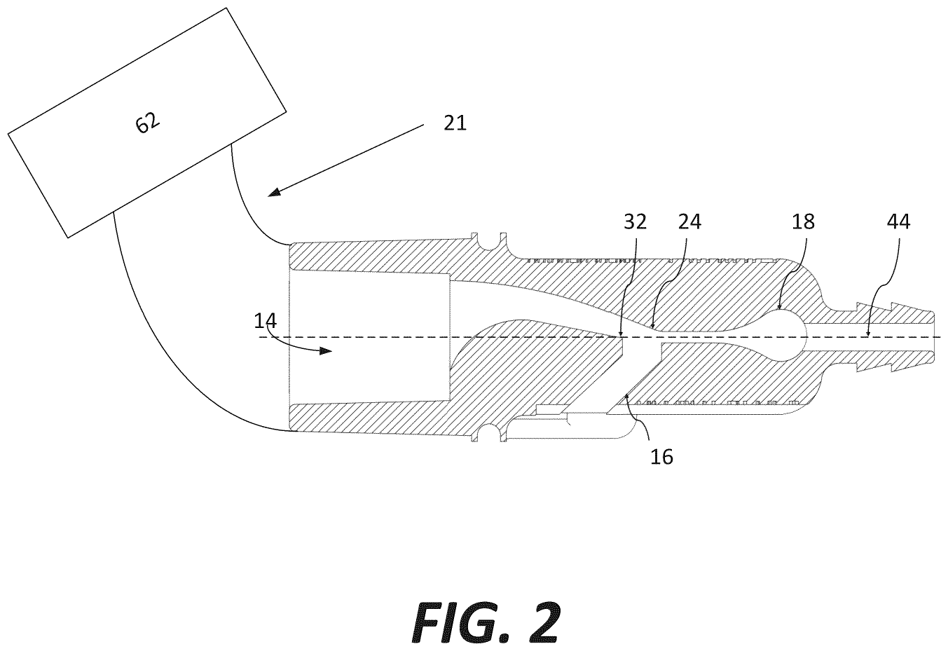

FIG. 2 schematically shows the valve 10 in accordance with illustrative embodiments of the invention. The fluidic valve 10 is configured to provide a number of advantages over the prior art, many of the advantages of which are described with reference to FIG. 2. It should be understood that some valves 10 may provide one or more of the various advantages listed herein. Various embodiments may include the following features and/or advantages:

As mentioned previously, illustrative embodiments do not require a specific radius of curvature 30 on the transition surface 24 of the device 10 (also referred to as the nozzle radius 30). Indeed, various embodiments do not require a radius of curvature 30, and may instead use a flat transition surface 24 (i.e., 0 nozzle radius 30 of curvature). In various embodiments, desirable valve 10 performance characteristics may be achieved with a wide variety of transition surfaces 24.

Illustrative embodiments offset 60 the splitter 32 from the nozzle axis 42. Prior art devices 10 that the inventors are aware of axially align the nozzle axis 42 with the center of the splitter 32. In contrast, various embodiments offset 60 the nozzle axis 42 from the splitter 32 (e.g., offset the nozzle axis 42 from the center of the splitter surface 38 or entirely from the splitter surface 38). Furthermore, in various embodiments the splitter 32 is configured so that the bifurcated channels 34 are not uniform. Various embodiments use the offset splitter 32 to bias fluid flow towards the patient side circuit 21 (e.g., fluidic tubing, endotracheal tube, and other fluidic connections that are downstream of the valve 10), which creates certain physiologically desirable characteristics.

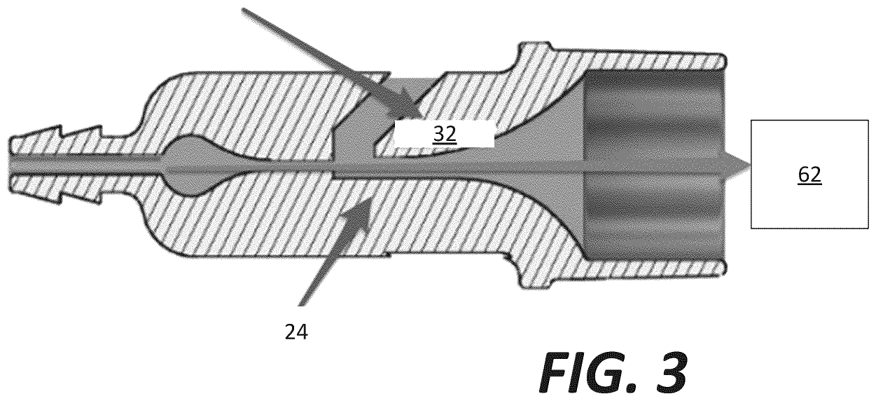

FIG. 3 schematically shows fluid flow through the device 10 in accordance with illustrative embodiments of the invention. Because the splitter 32 (also referred to as a diverter) is not directly in front of the nozzle 20, fluid flows towards the lungs of the patient 62. Thus, the flow is biased towards the lungs (i.e., towards the patient 62) by the position of the splitter 32, without the need for a specific curve on the transition surface 24. Furthermore, it is believed that the transition surface 24, which in FIG. 3 is also a step 26, helps bias the flow towards the patient outlet 14 (e.g., toward lungs). The small step 26 is believed to create a small region of low pressure that assists with pulling the fluid towards the transition surface 24.

Returning to FIG. 2, various embodiments also provide non-symmetrical fluid flow paths 34. Furthermore, the fluid flow paths 34 have different lengths and/or widths. The non-symmetrical fluid flow paths 34 having different dimensions provide desirable performance characteristics. For example, the asymmetrical branches help achieve clinically safe PIP, PEEP, RR and/or IE ratios.

Additionally, the valve 10 advantageously provides in-line fluid flow, such that the fluid flow paths of 34 the inlet 12 and/or the outlet 14 are parallel to (e.g., the same as) the main axis 44 of the device 10. In some embodiments, the nozzle axis 42 is the same as the longitudinal axis of the device 10. The longitudinal axis of the device 10 may also pass through the inlet 12. In some embodiments, the various components are co-planar (i.e., they share a common plane). Accordingly, flow is not required to enter the device 10 orthogonally and the form factor of the device 10 creates a compact, simple, streamlined device 10, free of parts that may catch and hook on things.

Various embodiments include a fluid expansion zone 18 distal to the inlet 12 of the device 10. The inventors believe that the fluid expansion zone 18 help cause turbulent flow, which in contrast to prior art expectations, increases performance stability. Accordingly, in various embodiments, the device 10 is configured to produce turbulent fluid flow, and to control turbulent fluid flow. This is contrast to other prior art devices 10, which aim to reduce turbulent flow.

Various embodiments are configured to receive an air input of between about 3 psi to about 5 psi and to provide between about 0 cmH20 and about 40 cmH20 to the patient.

As best shown in FIG. 1C, a variety of parameters of the valve 10 may be tuned to achieve specific performance characteristics for treating a patient (e.g., a human patient). For example, the patient throttle area 50, channel depth 52, and nozzle width 46 may be adjusted to control PIP for desired clinical outcomes.

Furthermore, the exhaust throttle area 58 and/or exhaust 16 leg angles may be adjusted to control PEEP and IE ratio for desired clinical outcomes. In particular the exhaust angle of the outlet leg 54 and the exhaust angle of the inlet leg 56 may be adjusted.

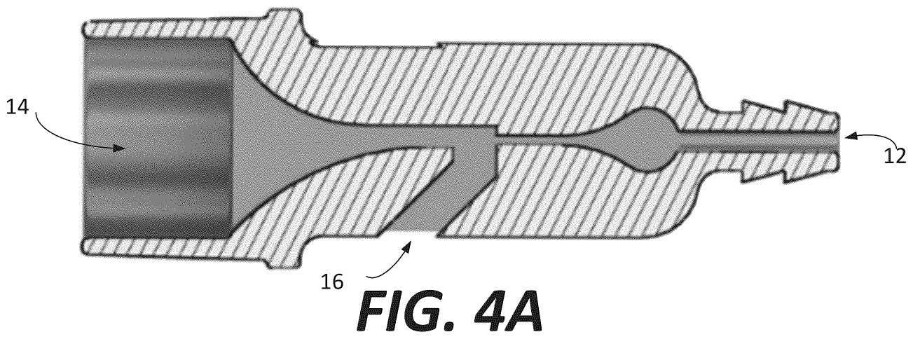

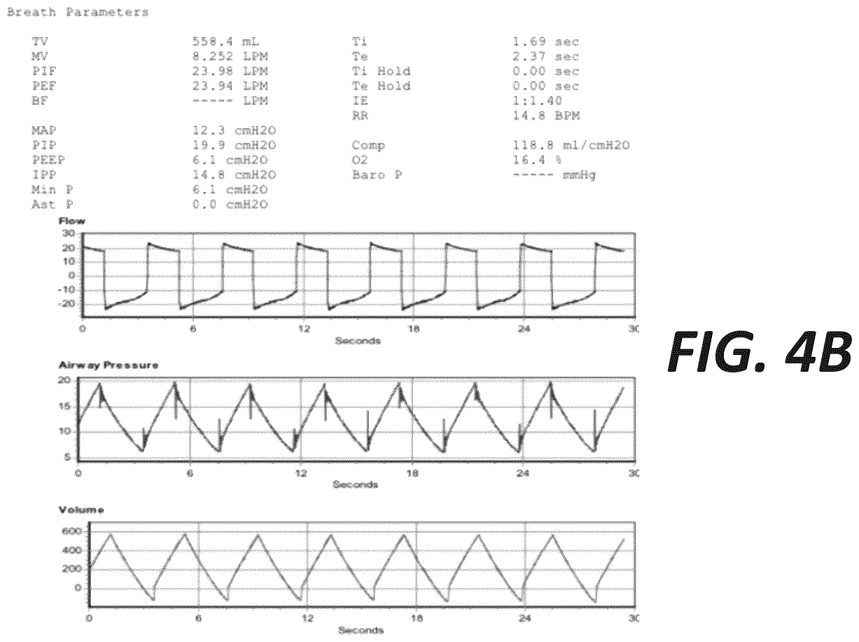

FIG. 4A schematically shows a fluidic valve 10 in accordance with illustrative embodiments of the invention. FIG. 4B schematically shows an example of air flow characteristics and breath parameters achieved through an exemplary valve 10 of FIG. 4A. As can be seen, the transition surface 24 may be a flat step 26. Additionally, as shown, various embodiments may position the diverter such that it does not intersect the nozzle axis 42. It should be understood that the air flow characteristics shown are an example and are not intended to limit various embodiments of the invention.

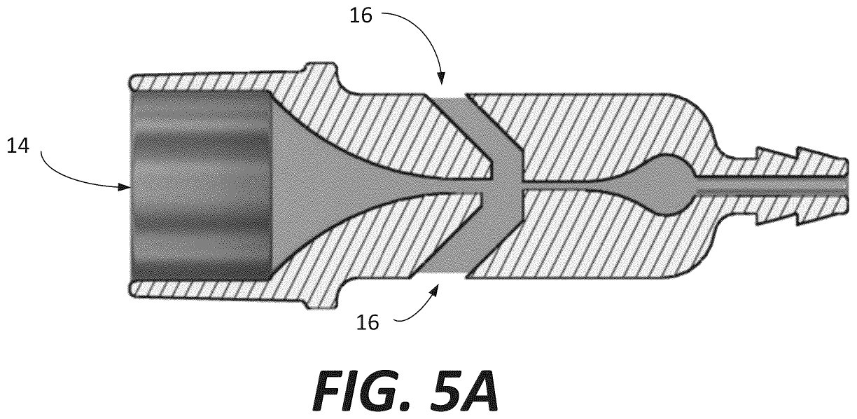

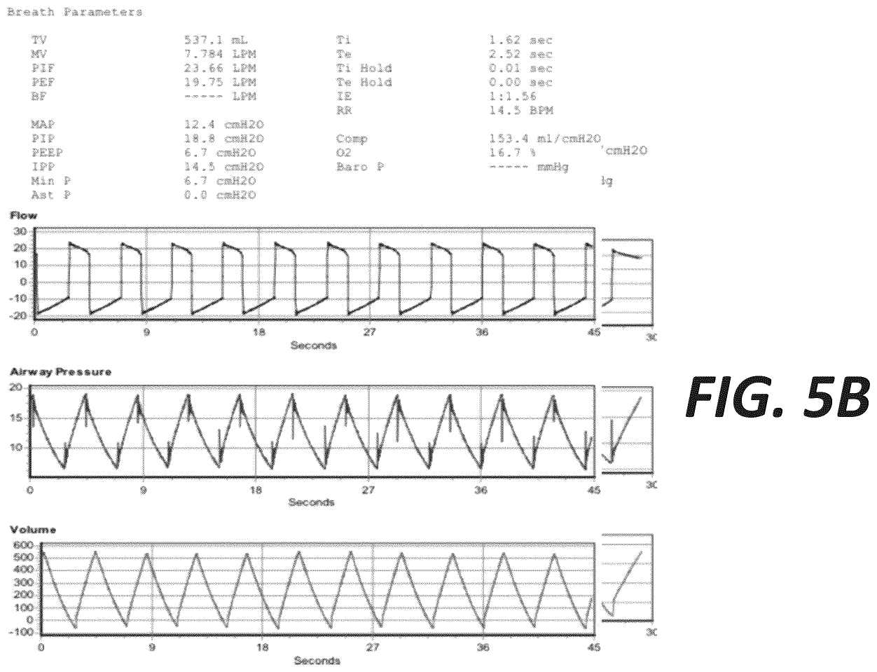

FIG. 5A schematically shows a fluidic valve 10 in accordance with illustrative embodiments of the invention. FIG. 5B schematically shows an example of air flow characteristics and breath parameters achieved through an exemplary valve 10 of FIG. 5A. As shown, various embodiments may have a plurality of exhausts 16. It should be understood that the air flow characteristics shown are an example and are not intended to limit various embodiments of the invention.

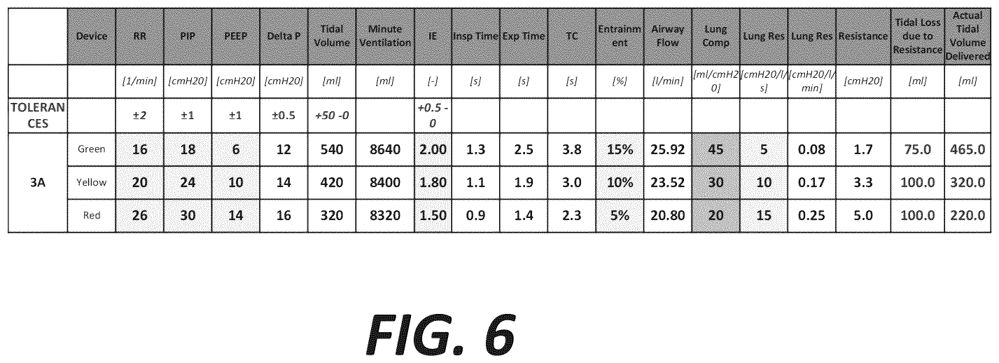

FIG. 6 schematically shows various performance parameters achieved by illustrative embodiments. It should be understood that the performance parameters shown are an example and are not intended to limit various embodiments of the invention.

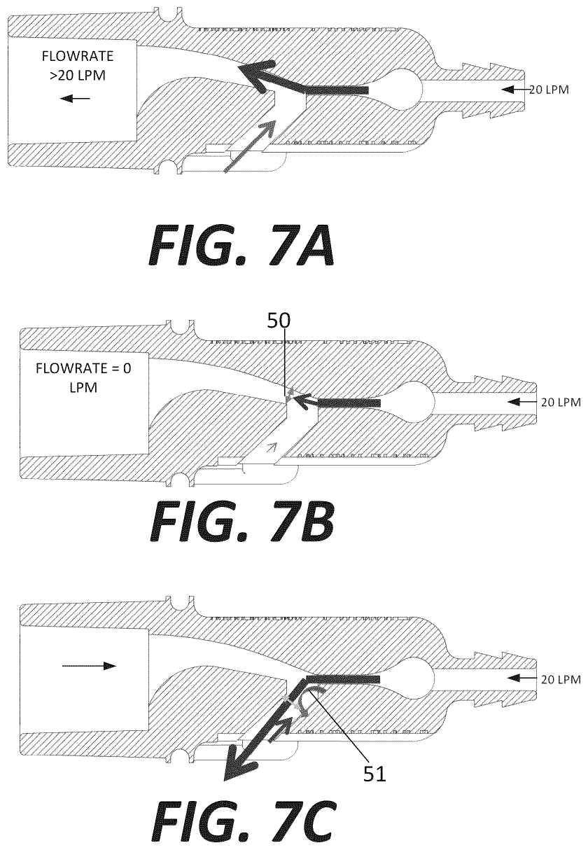

FIGS. 7A-9 are used to discuss the impact of various embodiments of the valve 10 on respiratory settings. Those skilled in the art understand these terms. However, FIGS. 7A-9 are provided to describe the inventors' belief as to how the valve 10 impacts these respiratory settings. Furthermore, FIGS. 7A-9 may show exemplary values, such as 20 LPM, which are not intended to limit various embodiments, but are instead shown for the sake of example.

FIGS. 7A-7C schematically illustrate the influence of the valve 10 on PIP in accordance with illustrative embodiments of the invention. Generally, PIP (Peak Inhalation Pressure) is fairly-well defined and correlates to the nozzle 20 cross sectional area (nozzle width 46×channel depth) and the motive flow rate. In this example, FIGS. 7A-7C show a flow rate coming into the inlet 12 (e.g., 20 LPM). It should be understood that the reference to 20 LPM is for discussion purposes only, and not intended to limit various embodiments. Various embodiments generate an air jet velocity of around 100 m/s (device 10 dependent).

As shown in FIG. 7A, the jet is held against the transition surface 24 until the patient backpressure rises to the point that the flow stalls to zero (as shown in FIG. 7B) in the patient throttle 50 cross section (shown in green). Then, the fluid flows to the exhaust 16 (as shown in FIG. 7C). At the point of the flow switching, the stagnation pressure generated in the patient throttle 50 cross sectional area is known as PIP.

PIP may be measured using a patient monitoring device data either from a patient airway manometer or from testing equipment which records this data as well as airflow. In FIG. 7C, the backflow varies throughout the exhaust phase and does not have a defined flipping flow rate back to inhalation cycle. Instead, the flow rate is a function of pressures indicated by the circular blue arrow 51 holding the jet in place.

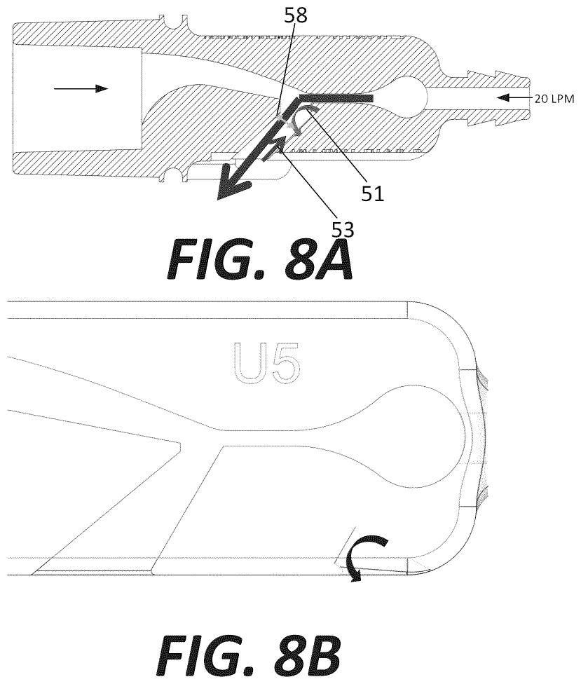

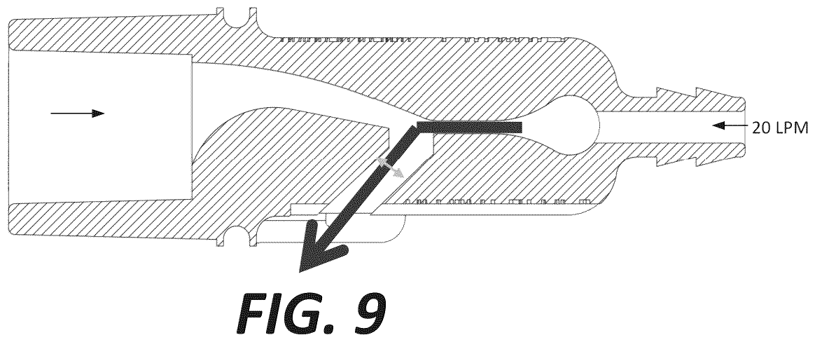

FIGS. 8A-8B schematically illustrate the influence of the valve 10 on PEEP in accordance with illustrative embodiments of the invention. Generally, PEEP (Positive End Expiratory Pressure) is more complex to tune and relies on more geometrical aspects. As the fluid flows to the exhaust 16 port, the low-pressure recirculation ‘bubble 18’ (blue 51) holds the jet until sufficient energy has dissipated and the ‘bubble 18’ or vacuum lock can be broken by turbulent air flowing back 53 (purple). Fluid then flows back towards the outlet 14 and towards the patient, and the process shown in FIGS. 7A-7C is repeated for another breath cycle.

In the embodiment shown in FIG. 8B, the bubble 18 region, and the collapse thereof, can be controlled directly using a variable set screw. This allows PEEP to be controlled to a wide range of values.

In various embodiments, PEEP is impacted by:
the angle that the main jet is deflected
the exhaust throttle 58 sectional area
the ‘bubble 18’ region geometry
the angle of the exhaust 16 port legs (not FIG. 8B has a diverging exhaust 16 and no nozzle outlet step 26)

FIG. 9 schematically illustrates the influence of the valve 10 on IE ratio settings in accordance with illustrative embodiments of the invention. The IE (Inhale Exhale) Ratio is the ratio of inhale duration to exhale duration. For example, an IE of 1:2 means the exhale portion is twice as long as the inhale portion during a single breath. The inhale portion of the breath time is essentially defined by the physiological requirements set out by the physicians during device 10 design.

Various embodiments may be tuned to adjust the following parameters:
RR (Respiration Rate)
TV (Tidal Volume)
MV (Minute Volume)=RR×TV
Inhale time

The orange arrow indicating exhaust throttle 58 cross sectional area at in part modulates the exhalation rate. The more convoluted this path, the greater the resistance is, and thus the larger the IE ratio (the slower the exhale). This dimension also controls to some degree the PEEP values so the dimensions in this region need to be adjusted together.

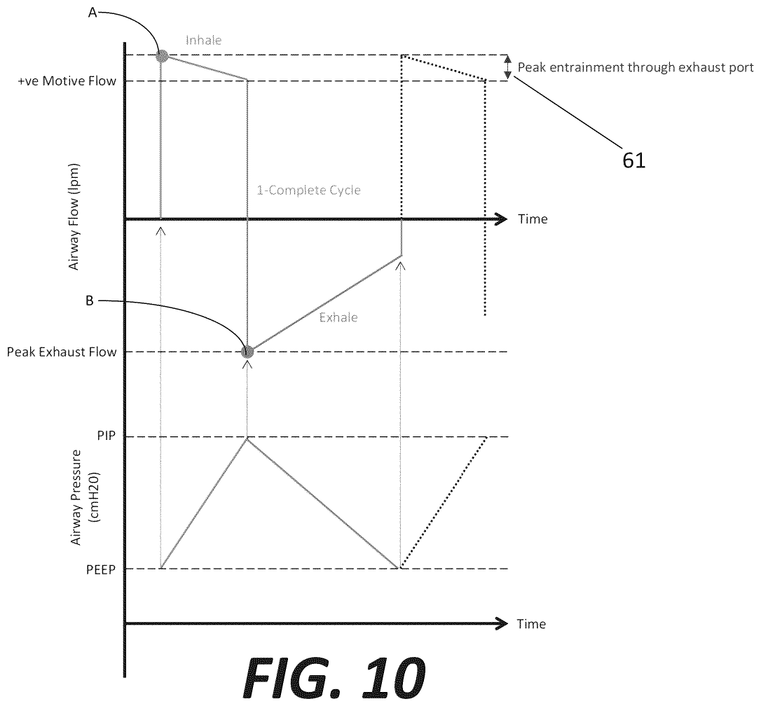

FIG. 10 schematically shows airway flow and airway pressure waveforms when the valve 10 is in use in accordance with illustrative embodiments of the invention. Starting from the BLUE dot (point A), the inhale stage begins. The full motive flow (i.e., the inlet 12 flow rate provided to the device 10 of clean gas (air/oxygen)) preferentially attracts to the transition wall and flows towards the patient outlet 14. In some embodiments, the attraction to the patient side wall may be caused by to the “Coanda Effect,” where the low pressure generated by the high nozzle 20 velocities results in the fluid jet tending to be directed towards the patient wall. However, various embodiments may be configured to operate without the use of the coanda effect. Ambient air is entrained through the exhaust 16 port during inhale. The peak entrainment occurs the instant the inhale stage begins. As the inhale stage proceeds, the entrainment gradually decreases until the point of switching to exhale, where the entrainment value is approximately zero.

Patient exhale starts when the jet flips from the patient outlet 14 to the exhaust 16. The inventors believe the point of switching occurs when the patient airway flow equals the motive flow rate. This point of switching can be viewed as an internal energy balance. For example, when the potential+kinetic energy in the incoming flow equals the potential+kinetic energy stored in the patient airway then the device 10 switches to the exhale stage.

Described another way, the device 10 operates in what may be considered an incompressible flow regime, and thus, air coming into the device 10 is equal to air moving out of the device 10. Thus, if the patient air flow rate slows down to the same as that of the motive flow rate, then the air switches to the exhale stage. Thus, in various embodiments, the inventor suspect, but have not confirmed, that the main jet is deflected towards the patient outlet 14 due to “coanda” effect, but that the switching time (e.g., the point of switching B in FIG. 10) is defined by the internal energy balance. This energy balance is calculated by the Bernoulli equation and is defined at the “patient throttle 50” cross-sectional area.

Continuing the operation from the GREEN dot (point B): after the jet is deflected towards the exhaust 16 port, the patient airway air flow reverses and discharges (together with the motive flow) out of the exhaust 16.

The peak exhaust 16 flow rate is a function of:
the internal resistances of the valve 10, defined by the geometry,
the patient airway resistances, and
the patient airway pressure,

As the exhaust 16 stage proceeds, the patient airway pressure drops which results in the total exhaust 16 flow rate decreasing.

As described previously, the exhaust 16 leg side differs to that of the patient side. This part of the cycle seems to rely on the low-pressure recirculation ‘bubble 18’ (shown as a red arrow 61 in FIG. 10) that is formed by the jet velocity as well as the valve 10 geometry in this region. The stronger this ‘bubble 18’ is, the lower the PEEP pressure.

As the patient airway flow decreases, the less the ‘bubble 18’ is pushed into the recirculation region up until the point that this bubble 18 ‘pops’ and the jet can break free back to the patient outlet 14 to start the inhalation cycle again.

Various embodiments described herein advantageously are configured to automatically transition the patient from inhalation to exhalation, and vice-versa. The device 10 may accordingly be said to oscillate between the fluid flow channels that lead to the outlet 14 and to the exhaust 16 when coupled with the patient (e.g., a patient who's breathing is entirely controlled externally by a ventilator). This is in contrast to some other embodiments that may operate as a switch having two or more stable states (e.g., bi-stable). Such bi-stable switching devices 10 require an external trigger (e.g., an external pressure applied from a patient initiating an inhalation or exhalation) to switch states (i.e., to switch flow paths). In various embodiments, the device 10 is configured so that fluid flow automatically returns to the patient outlet 14 after it is diverted to the exhaust 16. Thus, the device 10 may be described as a fluid oscillator.

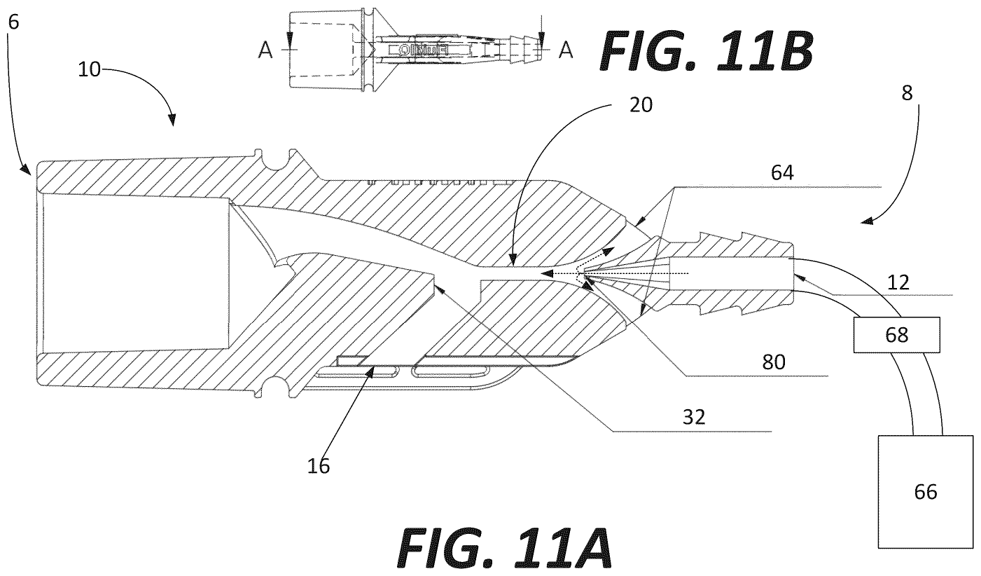

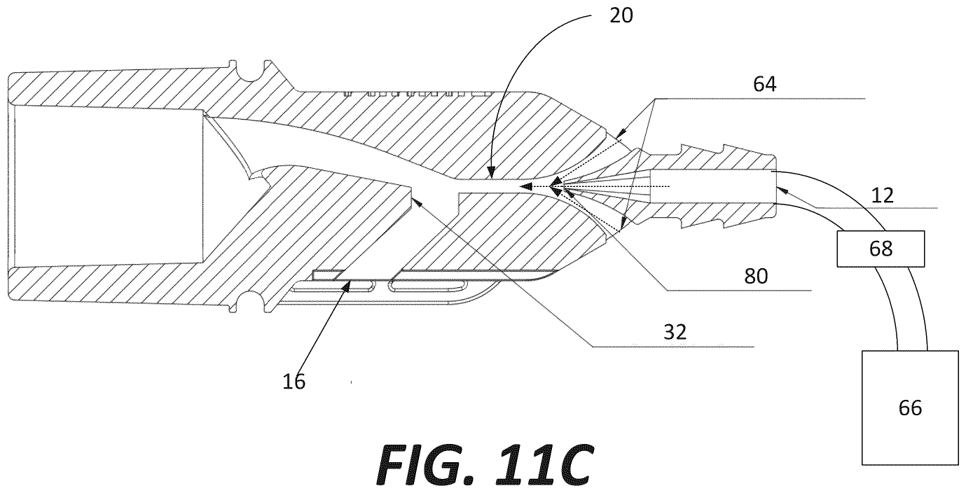

FIGS. 11A-11C schematically show the device 10 in accordance with illustrative embodiments of the invention. The device 10 is configured to reduce input fluid source 66 (e.g., oxygen or other gas) concentration. Although described with reference to FIGS. 11A-11C, it should be understood that all of the various embodiments described previously may be coupled to a fluid source 66. The device 10 has a distal end 6 that is closer to the patient circuit 21, and a proximal end 8 that is closer to the fluid source 66 and/or inlet 12.

As shown in FIGS. 11A-11C, some embodiments may not include the fluid expansion zone 18. Alternatively, or additionally, various embodiments may include a dilution nozzle 80. In various embodiments, fluid (e.g., 95% medical grade oxygen) from the fluid source 66 passes through the dilution nozzle 80 and is diluted with ambient air (approximately 21% oxygen). The dilution nozzle 80 may also be referred to as an inlet nozzle 80 in some embodiments. Thus, fluid passing through the dilution nozzle 80 (e.g., in a direction from the proximal end 8 towards the distal end 6) may be diluted with air that is received through a port 64.

Accordingly, one or more port(s) 64 may be adjacent to the dilution nozzle 20. Otherwise, the device 10 of FIGS. 11A-11C may include some or all of the features of various embodiments described herein. The ports 64 may be referred to as “outlet-side port 64” or “exhaust-side port 64” to indicate a relative position with respect to a cross-sectional view (e.g., an outlet-side port 64B is closer to the outlet 14 than an exhaust-side port 64A). In a similar manner, the device may have an outlet-side surface 74 and an exhaust-side surface 76. In some embodiments, the ports 64 may be positioned distally relative to the dilution nozzle 80 (e.g., as shown in FIGS. 11-14). However, in some other embodiments, the ports 64 may be positioned proximally relative to the dilution nozzle 80.

The one or more ports 64 may provide a number of advantages. For example, the ports 64 may operate a safety mechanism that limits pressure on the patient 62 side. In particular, the device 10 may restrict the input gas (e.g., oxygen) flow rate through the device nozzle 20. As the fluid flow rate through the inlet 12 is increased beyond a given threshold, the path of least fluid resistance becomes the one or more port(s) 64. Accordingly, fluid flow is directed out of the ports 64, which may be tuned to provide an upper limit on the fluid flow rate through the device nozzle 20.

In contrast, the one or more exhausts 16 of the device 10 may be downstream of the device nozzle 20 and assist with automatic transitioning between the fluid flow paths, as described above (e.g., between the outlet 14 or the exhaust 16 (s)). In various embodiments the port(s) 64 are upstream of the device nozzle 20. The port(s) 64 may assist with controlling and capping the total amount of fluid flow passing through the device nozzle 20.

Another advantage of various embodiments includes a reduction in the amount of a source fluid (e.g., an oxygen source) that is used. In various embodiments, the fluid source 66 (e.g., provided by medical personnel) may have a given FiO2 percentage (e.g., emergency personnel have with them, such as 95% medical grade oxygen FiO2). Fluid sources 66 used by medical personnel tend to be higher concentrated oxygen (e.g., 95% FiO2). Depending on the desired patient side parameters that the device 10 outputs, the preferred or acceptable range of FiO2 may vary. Various embodiments use one or more ports 64 (also referred to as dilution ports 64 or suction ports) to dilute the fluid from the fluid source 66 using air from ambient (i.e., ambient air with approximately 21% oxygen concentration). Accordingly, the dilution ports 64 are configured so that air from the outside environment is brought into the device 10 to dilute the input fluid (e.g., the fluid through the inlet 12). The diluted fluid passes through the device nozzle 20 towards the patient.

In general, the fluid source 66 and associated equipment (e.g., a regulator 68, fluidic tubing, etc.) is configured to provide a given flow rate of the fluid into the device 10. For the sake of discussion, the fluid source 66 may be 100% oxygen. However, it should be understood that any concentration of oxygen may be used, and indeed, some embodiments may use non-oxygen containing fluid sources 66.

In the absence of the ports, the flow rate from the fluid source 66 remains constant as fluid flows transitions from the patient outlet 14 to the exhaust 16. Thus, even when fluid is flowing towards the exhaust 16, the input fluid flow rate from the source remains constant. The fluid flow is constant in various embodiments because the fluid flow acts as a controlling flow that switches the flow path from the outlet 14 to the exhaust 16 and vice-versa. Accordingly, in various embodiments, as the patient 62 exhales, fluid from the source 66 is wasted. Illustrative embodiments thus reduce higher concentration fluid source 66 using ambient air, and provide the mixed/diluted air to the patient 62. In some embodiments, the ports 64 may substantially dilute the fluid from the fluid source 66 (e.g., dilute the fluid from the fluid source 66 by 15%-60%).

Various embodiments may use, for example, an oxygen D cylinder (i.e., 340 liters) as the source. Depending on the amount of time that the patient 62 is fluidly coupled to the fluid source 66, there is a concern that the oxygen source may run out (e.g., after 15 to 20 minutes). The dilution ports 64 are configured such that the main flow from the inlet 12 entrains fluid flow from the outside environment. For example, the flow rate through the device 10 may be configured to be about 20 liters per minute. Without the dilution ports, 20 liters per minute are obtained from the source. However, illustrative embodiments having the dilution portions may drop the flow rate from the source (e.g., to 12 LPM) and entrain ambient air through the dilution portions (e.g., to obtain the remaining 8 liters per minute, providing a total flow rate of 20 lpm). Illustrative embodiments thus reduce the fluid flow rate from the fluid source 66 and extend the useful life of the fluid source 66. This is particularly advantageous in emergency situations (e.g., where smaller sources of fluid are available).

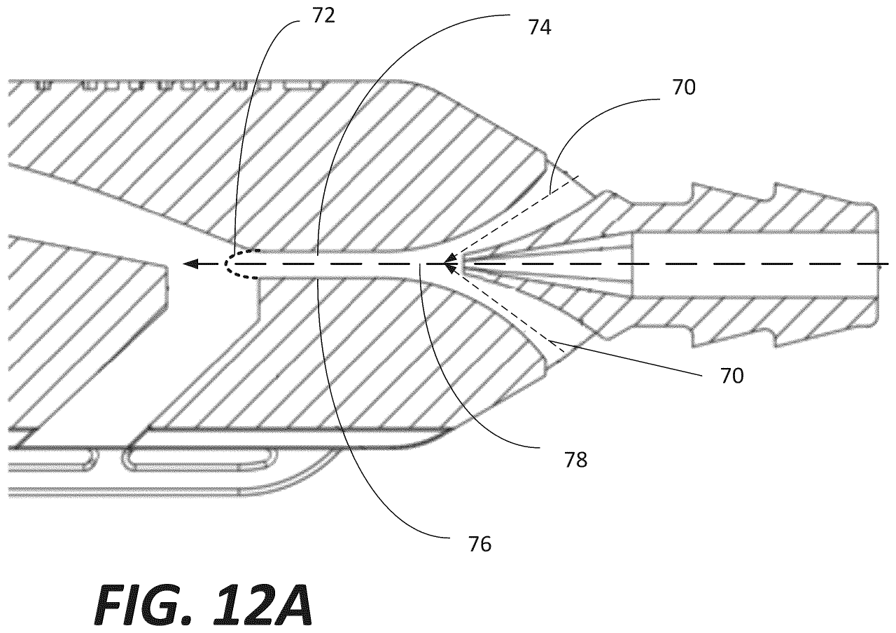

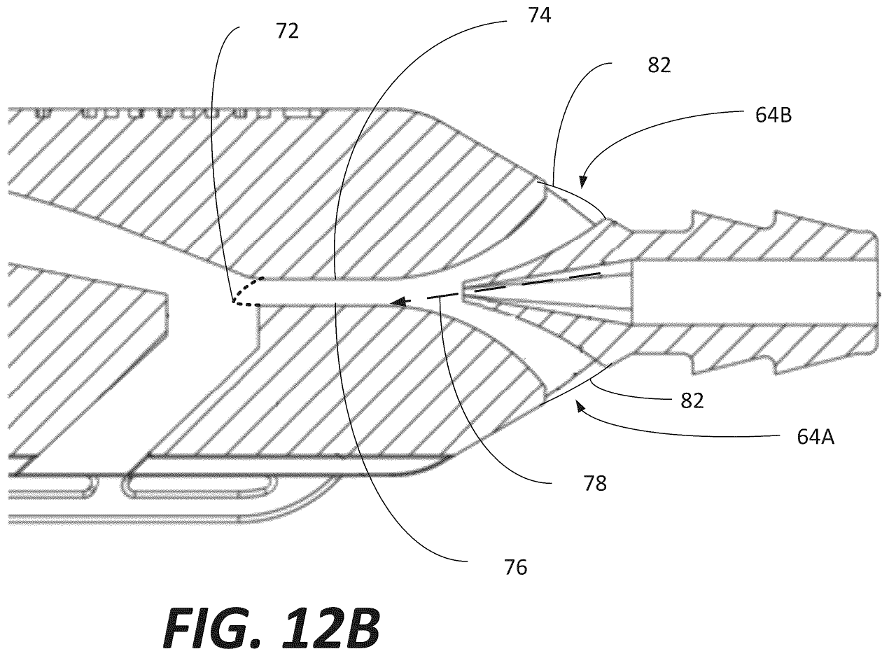

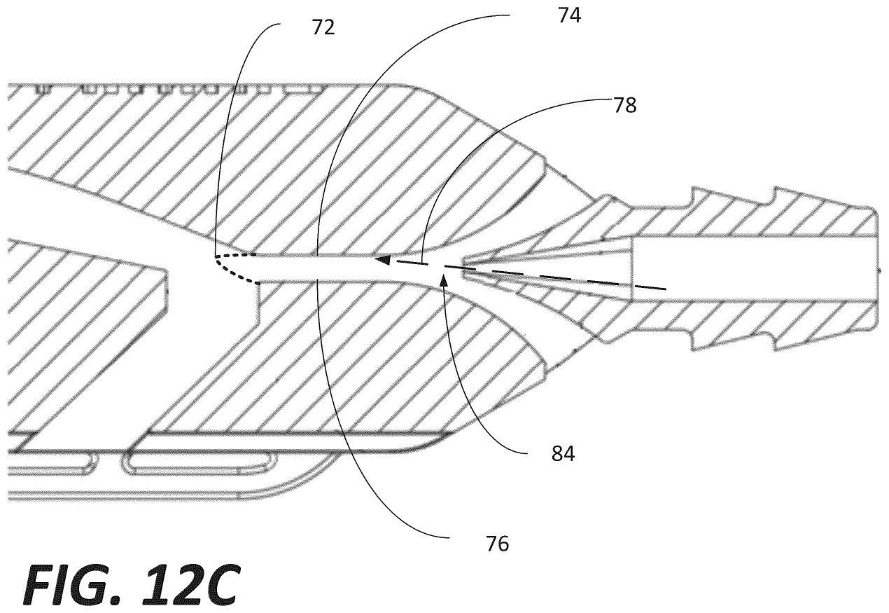

FIGS. 12A-12C schematically show various configurations of biased air flow through the inlet 12 in accordance with illustrative embodiments of the invention. FIG. 12A schematically shows air flow coming through the inlet 12 and being substantially aligned with the device nozzle axis 42. Air flow may also be entrained 70 from outside of the device 10 through the dilution ports 64. As shown, the entrained airflow 70 joins the primary fluid flow coming in through the inlet 12. FIGS. 12B and 12C do not show the entrained flow 70, but it should be understood that these embodiments also entrain airflow 70 from ambient or from another source (unless the port 64 is blocked).

Without wishing to be bound by any theory, the inventors believe, but have not confirmed, that the suction effect through the ports 64 is caused by a relatively low pressure at the outlet 14 of the dilution nozzle 20, relative to the air outside the device 10. This low pressure is generated by the high velocity exiting the dilution nozzle 20. The relatively higher pressure outside the device 10 causes ambient air to come in to the relatively low pressure area in the device 10, where it joins the fluid jet coming from the inlet 12 (e.g., from the fluid source 66).

In various embodiments, FIG. 12A represents a flow of fluid that is substantially parallel with the device nozzle axis 42. A profile 72 of the fluid flow is schematically shown in FIG. 12A. As shown with references to FIGS. 12A-12C, the profile 72 may impact the biasing of the fluid flow in various ways. The inventors discovered that the fluid flow profile 72 also impacts the PIP and/or PEEP of the device 10, along with other end-patient parameters (e.g., IE ratio, etc.).

FIG. 12B schematically shows air flow coming through the inlet 12 that is heading towards the exhaust-side wall 76 (at least in the view shown). This ultimately changes the position of the fluid flow relative to the nozzle 20. A profile 72 of the fluid flow is schematically shown in FIG. 12B. The primary direction 78 of the airflow coming into the device 10 is shown pointing towards the exhaust-side wall 76. Illustrative embodiments may provide this direction of airflow by changing the relative orientation of the inlet 12 and/or the dilution nozzle 80 (e.g., by rotating the position of the inlet 12 relative to the device 10), such that the fluid flow points substantially towards the exhaust-side wall 76. Additionally, or alternatively, the fluid flow direction may be changed by blocking one or more of the dilution ports 64. For example, some embodiments may block the exhaust-side port 64A. The inventors believe this creates a region of low pressure which biases the flow towards the exhaust-side wall 76. Some embodiments may include a movable closure 82 (e.g., slidable with a finger of the medical practitioner) over one or more of the ports 64. By closing one or more ports 64, the performance of the device 10 may be adjusted.

FIG. 12C schematically shows the primary direction of air flow 78 coming through the inlet 12 is heading towards the outlet-side wall 74. This ultimately changes the position of the fluid flow relative to the nozzle 20. A profile 72 of the fluid flow is schematically shown in FIG. 12C. The primary direction of the airflow 72 coming into through the nozzle 20 is shown pointing towards the patient-side outlet 14. Illustrative embodiments may provide this direction of airflow by changing the relative orientation of the inlet 12 and/or the dilution nozzle 80 (e.g., by rotating the position of the inlet 12 relative to the device 10), such that the fluid flow points substantially towards the patient-side wall 74. Additionally, or alternatively, the fluid flow direction may be changed by blocking one or more of the dilution ports 64. For example, some embodiments may block the patient-side port 64B. The inventors believe this creates a region of low pressure which biases the flow towards the patient-side wall. Some embodiments may include a movable closure 82 (e.g., slidable with a finger) over one or more of the ports 64. By closing one or more ports, the performance of the device 10 may be adjusted.

By biasing the fluid flow towards the patient-side wall 74, illustrative embodiments increase PIP and/or PEEP. On the other hand, by biasing the fluid flow towards the exhaust-side wall 76, illustrative embodiments decrease PIP and/or PEEP. The inventors further discovered that blocking of the ports 64 has differing magnitudes of effect on overall device 10 characteristic.

Based on the above disclosure, it should be apparent that various embodiments may adjust fluid flow dynamics by blocking dilution ports 64 and/or by adjusting the relative orientation of the dilution nozzle 80 relative to the device nozzle 20. In various embodiments, the dilution nozzle 80 and device nozzle 20 share a common plane (e.g., even when the inlet 12 is rotated, such that the direction of the airflow is changed but the fluid flow remains substantially co-planar).

Some embodiments may adjust the inlet 12 so that it is not in-line with the device nozzle axis 42 (e.g., perpendicular), such that the primary direction of incoming airflow is perpendicular to the device nozzle axis 42. The inventors believe that such a configuration is not preferred because the primary direction of airflow would not be directed towards the device nozzle 20, but instead, hit some other portion (e.g., a wall of the atrium 84 of the device 10). The back pressure created by flow hitting a wall and then expanding outwardly would be likely to cause inconsistent performance through the ports, and instead cause the ports 64 to operate as exhausts 16 instead of suction/entrainment ports 64.

In various embodiments, when:
(1) Both ports 64 open, the device 10 functions smoothly and consistently;
(2) The patient-side port 64 is blocked, PIP and PEEP increase;
(3) The exhaust-side port 64 is blocked, PIP and PEEP decrease.

It should be understood that the various flow profiles 72 and fluid flow directions shown herein are for discussion purposes and not intended to limit various embodiments. Indeed, the flow profiles 72 and fluid flow directions are shown to facilitate discussion, and not to imply that the profiles 72 or directions do not or cannot vary from what is shown.

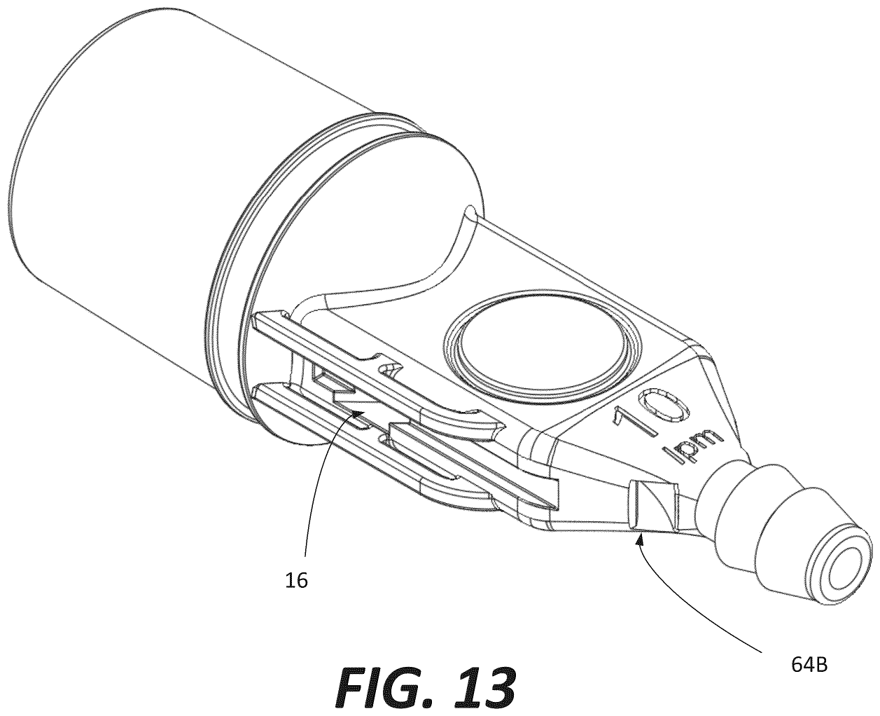

FIG. 13 schematically shows an external view of the device 10 in accordance with illustrative embodiments of the invention.

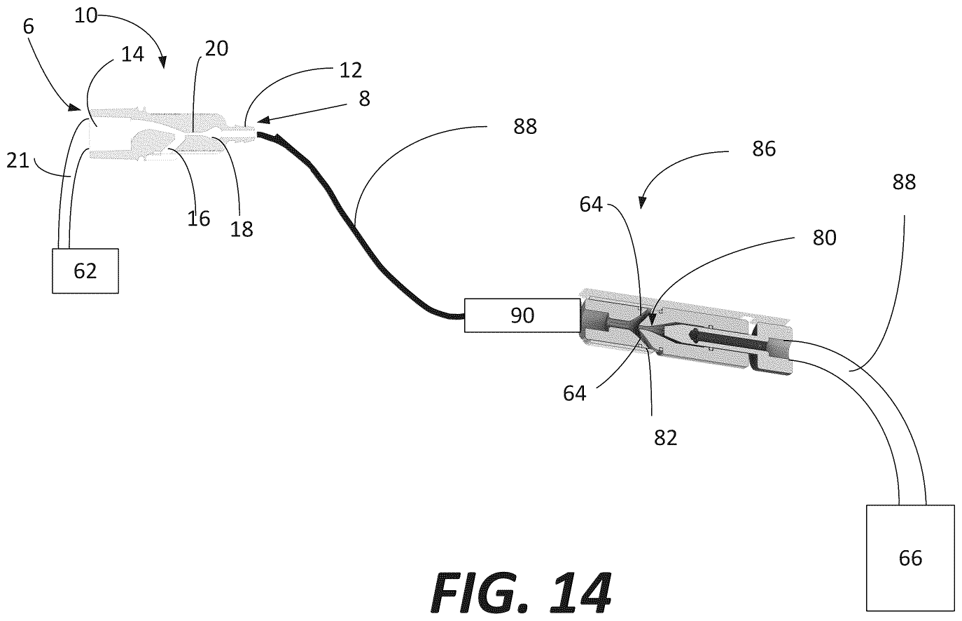

FIG. 14 schematically shows an alternative embodiment of the device 10 in accordance with illustrative embodiments. The device 10 shown in FIG. 14 may be identical to any of the devices 10 described herein, but may be further coupled with a dilution connector 86. Additionally, the dilution nozzle 80 may be coupled to the device 10 inlet 12 via a fluidic tubing 88 (e.g., with an optional flow meter 90). The dilution nozzle 80 may be configured to couple with the fluid source 66 (e.g., using standard fluidic tubing and connections). The flow meter 90 assists with setting the device 10 to achieve the desired flow rate.

In contrast to the embodiment shown in, for example FIGS. 12A-12C, the dilution nozzle 80 is in the dilution connector 86, which is not directly coupled with the device nozzle 20. Therefore, the direction of air flow is not as likely to impact device 10 performance as the embodiments shown in FIGS. 12A-12C. Instead, the airflow towards the device nozzle 20 is likely to be influenced by the geometry of the inlet 12 and/or inlet nozzle 20. In some embodiments, the fluidic device 10 inlet 12 may include a nozzle 20 configured to adjust the flow of air in the device 10, similar to the dilution nozzle 80 shown and described with reference to FIGS. 12A-12C (but not shown in FIG. 14).

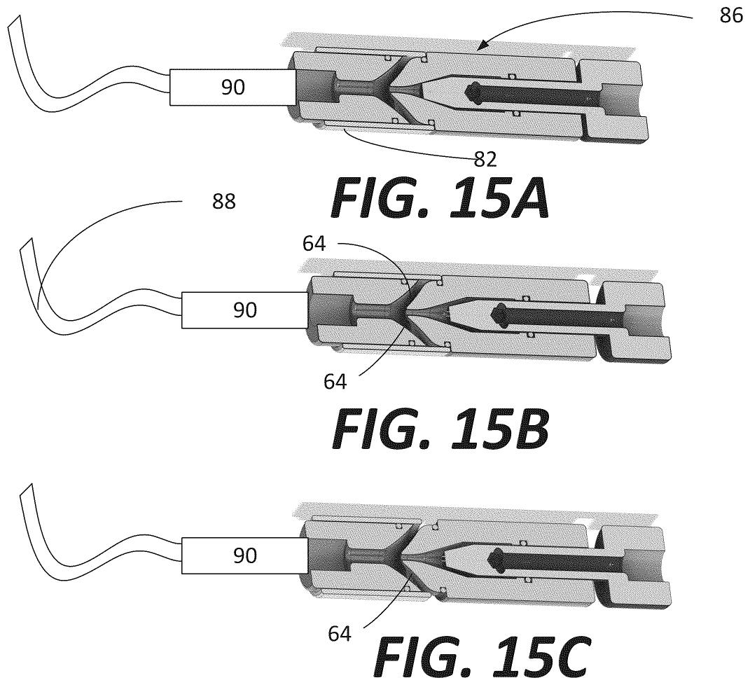

FIGS. 15A-15C schematically show the dilution connector 86 of FIG. 14 in accordance with illustrative embodiments of the invention. An adjustable closure 82 (e.g., a slider) on the top section may be adjusted to control the amount of fluid that is entrained into the tubing 88 that goes to the fluidic device 10. The devices 10 show the stages of adjustment that achieve a final desired FiO2 and flow rate.

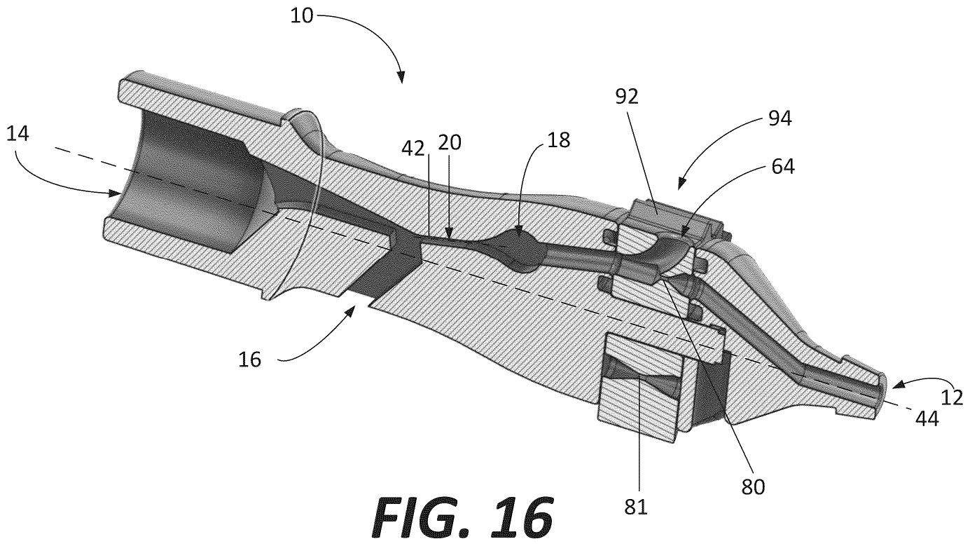

FIG. 16 schematically shows a cross-sectional view of an alternative embodiment of the device 10 in accordance with illustrative embodiments. The device 10 includes a nozzle switcher 94 that provides for rapid adjustment between diluted fluid (e.g., from fluid entrained through the port 64) and pure fluid that is received from the source 66 (i.e., without entrained air). Accordingly, in various embodiments, the device 10 may transition (e.g., rotatably) between the dilution nozzle 80 and a non-dilution nozzle 81. The dilution nozzle 80 is coupled with the port 64. Whereas the non-dilution nozzle 81 does not include fluid access to the port 64. The user may easily switch between the dilution nozzle 80 and the non-dilution nozzle 81 by manual manipulation, e.g., by rotating the nozzle 80, 81 using rotatable transition features 92 (e.g., projections, textured surface, etc.), such that a fluid flow path of the inlet 12 aligns and fluidly couples with the nozzle 80 or 81.

Accordingly, some embodiments may transition between the nozzles 80, 81 and/or may use movable cover 82 to assist with entrainment of air. Furthermore, although only two nozzles are shown 80, 81, it should be understood that a device may rotate through a number of different nozzles 80, 81 having a variety of different sized nozzles 80 and ports 64.

In some embodiments, the fluid flow path of the inlet 12 may be non-linear, as shown. Additionally, in some embodiments, the axis 42 of the device nozzle 20 may be non-parallel with the device axis 44. However, various embodiments include a planar flow path from the inlet 12 to the device nozzle 20.

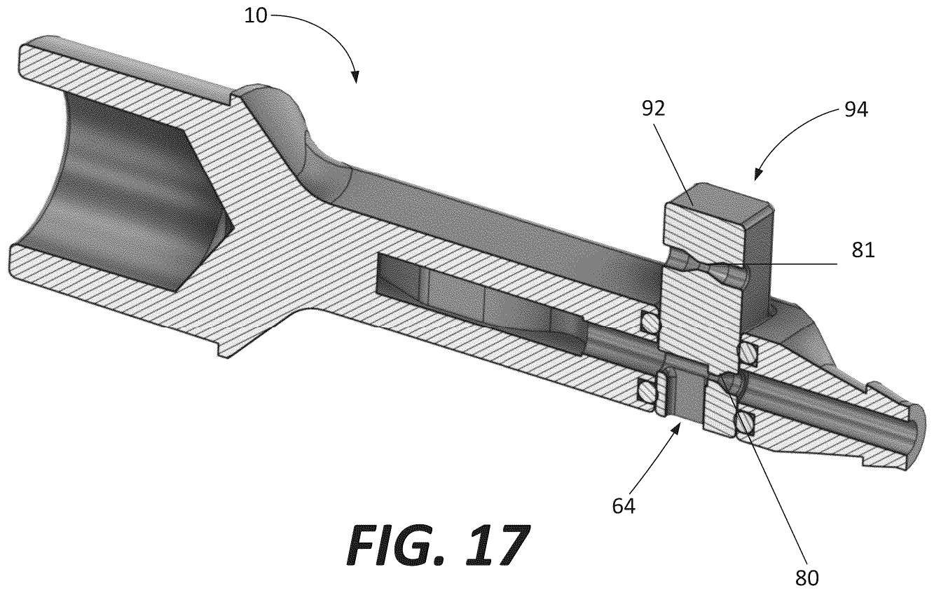

FIG. 17 schematically shows a cross-sectional view of an alternative embodiment of the device 10 in accordance with illustrative embodiments. Among other things, FIG. 17 shows the device having a planar flow path from the inlet 12 to the device nozzle 20. The device 10 includes a transition feature 92, which may be pushed to transition between the dilution nozzle 80 and the non-dilution nozzle 81. Accordingly, the dilution nozzle 80 or the non-dilution nozzle 81 may slide in-line (e.g., parallel) with the fluid flow path from the inlet 12.

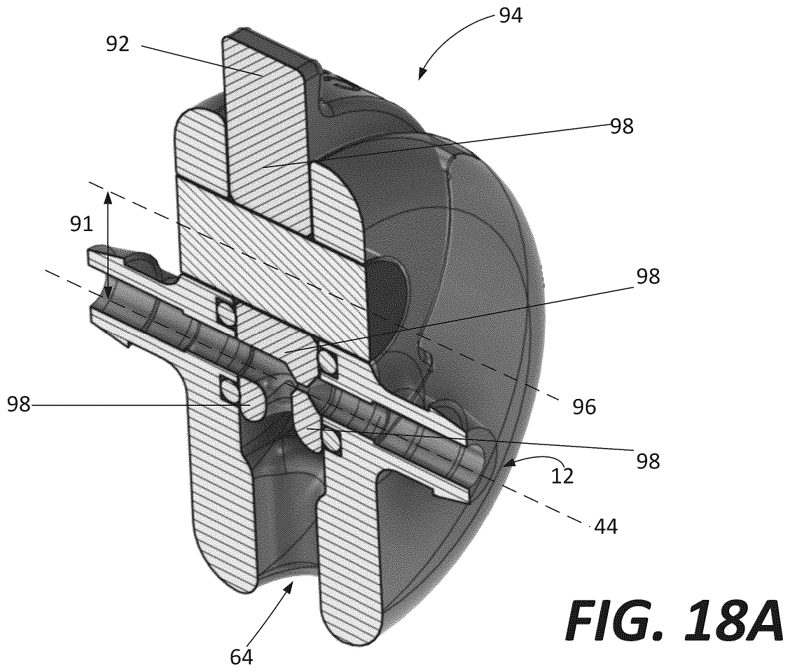

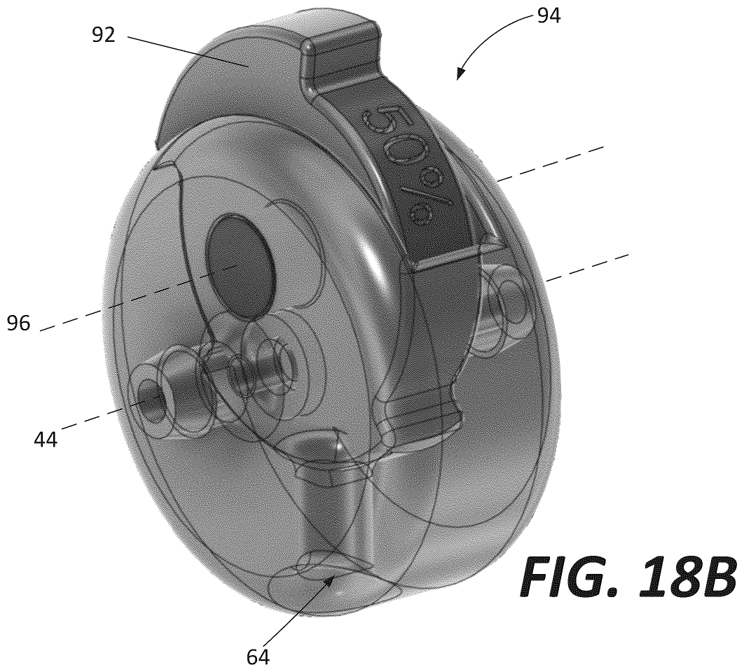

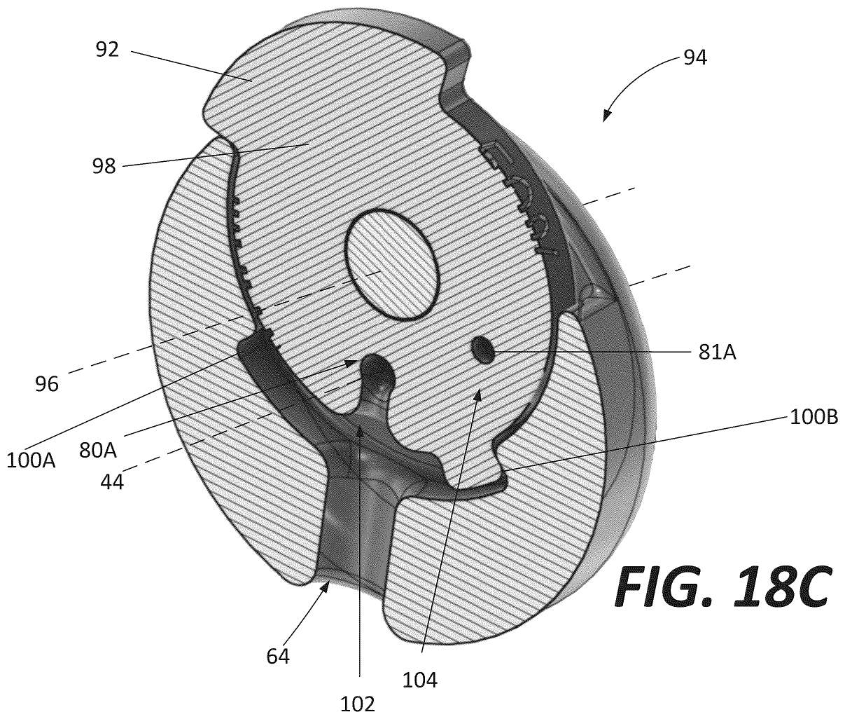

FIGS. 18A-18C schematically show details of a nozzle switcher 94 in accordance with illustrative embodiments. In various embodiments, the nozzle switcher 94 is integrated into the device 10 (e.g., as shown in FIGS. 16 and 17). However, the nozzle switcher 94 may be a separate piece that couples to the device 10. FIG. 18A schematically shows a cross-sectional view of the nozzle switcher 94 in accordance with illustrative embodiments. FIG. 18B shows a partially transparent view of the nozzle switcher 94 of FIG. 18A. In the view of FIG. 18B, the nozzle switcher 94 is rotated by 90 degrees relative to FIG. 18A. FIG. 18C schematically shows a sectional view of the nozzle switcher 94 of FIG. 18B. The switcher 94 may have an axis of rotation 96 that is offset 91 from the longitudinal axis 44 of the device 10. A rotating portion 98 is configured to rotate around the axis of rotation 96. Although not visible from this view, the switcher 94 allows for rotation through a variety of dilution nozzles 80 and non-dilution nozzle 81 while maintaining the flow path of the device 10 co-planar and substantially co-linear.

FIGS. 18B-18C schematically show the arrangement of the switcher 94 in an entrained flow configuration where the switcher 94 is in a first configuration (also referred to as a diluted configuration). In the diluted configuration, a vented passage 80A, having an opening 102 to the port 64, is aligned with the fluid flow channel of the device 10. The switcher 94 may be transitioned to a non-dilution configuration (not shown) where an unvented passage 81A, having a blocking element 104 for the port 64, is aligned with the fluid flow channel of the device 10. In particular, the rotating portion 98 is set so that a transition of the graspable feature 92 by the user causes a rotation of the rotating portion 98 that aligns the vented passage 80A or the unvented passage 81A with the fluid flow channel of the device 10. To switch from pure flow from the fluid source 66 to diluted flow, the user may simply rotate the switcher 94. It should be understood that although the switcher 94 shows 50% printed thereon, various embodiments are not limited to a dilution of 50%. Indeed, various embodiments may achieve a diluted oxygen concentration of any desired percentage (e.g., 55%).

In various embodiments, the switcher 94 includes an alignment feature 100 (e.g., wall 100A and a projection 100B) configured to align the passages 80A and/or 81A with the fluid flow channel of the device 10. Additionally, the alignment features 100 prevent over-rotation of the rotating portion 98. Accordingly, the user may easily and reliably transition the switcher 94 between the first configuration (e.g., where the device nozzle 20 is fluidly coupled with the port 64), and the second configuration (e.g., where the device nozzle 20 is fluidly uncoupled with the port 64). Furthermore, the transition may occur without misalignment of the passages 80A and/or 80B with the fluid flow channel of the device 10 because of the alignment features 100 (e.g., user rotates rotating portion 98 until it can no longer rotate). The device 10 may also lock in the first configuration and/or the second configuration, such that a threshold force must be applied to transition the switcher 94 (e.g., it is not easily dislodged from the set configuration).

In various embodiments, the axis of rotation 96 is advantageously offset 91 such that the passages 80A and 81A align with the central axis 44 of the device 10 when rotated. Accordingly, the device 10 may keep a substantially planar fluid flow channel. Additionally, illustrative embodiments may use a small angular rotation (e.g., of 45 degrees or less) to transition between the first configuration and the second configuration. Of course, various embodiments may require different angular rotation for transitioning between configurations (e.g., greater than 45 degrees).

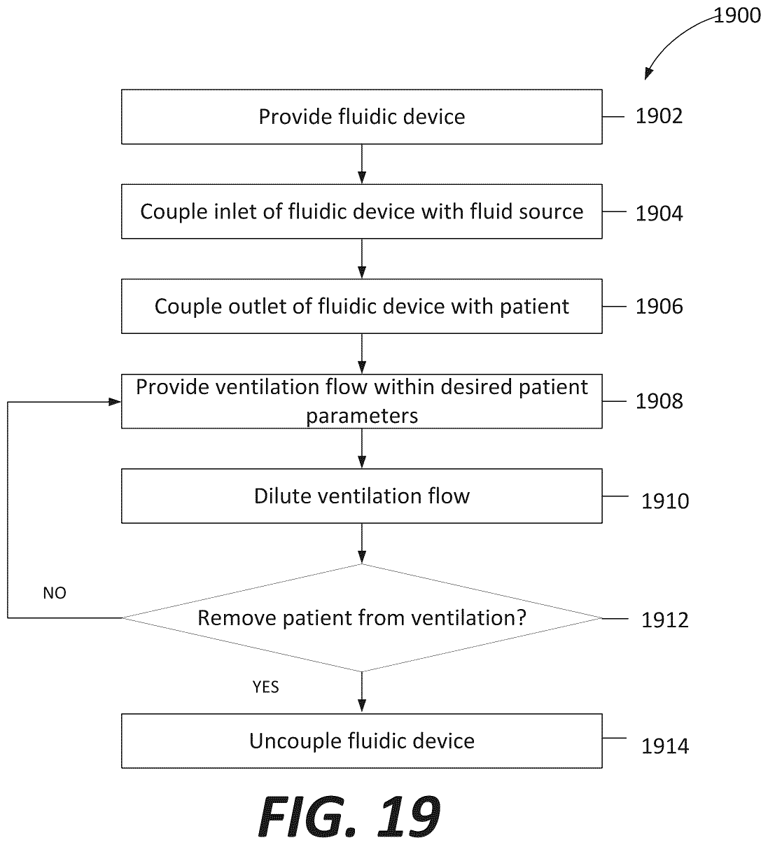

FIG. 19 shows a process of mechanical ventilation using the device 10 in accordance with illustrative embodiments of the invention. It should be noted that this method is substantially simplified from a longer process that may normally be used. Accordingly, the method shown in FIG. 19 may have many other steps that those skilled in the art likely would use. In addition, some of the steps may be performed in a different order than that shown, or at the same time. Furthermore, some of these steps may be optional in some embodiments. Accordingly, the process 1900 is merely exemplary of one process in accordance with illustrative embodiments of the invention. Those skilled in the art therefore can modify the process as appropriate.

The process 1900 begins at step 1902, which provides the fluidic device 10 as described herein. Briefly, the device 10 includes the inlet 12 configured to receive fluid from a pressurized source 66 (e.g., a mechanical ventilator or pressurized tank). The device 10 also includes the outlet 14 configured to couple with the breathing circuit 21 of the patient 62 and the exhaust 16.

At step 1904, the inlet 12 of the device 10 is coupled with the fluid source 66 and/or the mechanical ventilator. At step 1906, the outlet 14 is coupled with the breathing circuit 21 of the patient 62. Steps 1904 and 1906 may occur in a different order than shown in FIG. 19.

At step 1908, ventilation flow is provided through the device 10 to achieve desired patient ventilation parameters. Advantageously, various embodiments are configured to automatically transition the patient 62 from inhalation to exhalation, and vice-versa. The device 10 may accordingly be said to oscillate between the fluid flow channels 34 that lead to the outlet 14 and to the exhaust 16 when coupled with the patient 62 (e.g., when breathing is entirely controlled externally by a ventilator). Additionally, or alternatively, some embodiments may operate as a switch having two or more stable states (e.g., bi-stable). Such bi-stable switching devices 10 require an external trigger (e.g., an external pressure applied from a patient initiating an inhalation or exhalation) to switch states (i.e., to switch flow paths). In various embodiments, the device 10 is configured so that fluid flow automatically returns to the patient outlet 14 after it is diverted to the exhaust 16. Thus, the device 10 may be described as a fluid oscillator. The device 10 may operate in a given mode based on the mode of the ventilator (e.g., controlled ventilation, assisted ventilation, supported ventilation).

In various embodiments, the device 10 may be configured to produce desired patient ventilation parameters. FIG. 10 schematically shows a variety of device 10 modulated patient parameters. For example, the device 10 may be configured to produce a target PIP of between about 18 cmH20 and 30 cmH20 in the patient circuit 21. Air then flows out of the exhaust after the target PIP is reached, until a PEEP of between about 6 cmH20 and 14 cmH20 is achieved in the patient circuit 21. The steps of flowing air out of the outlet 14 and out of the exhaust 16 define a patient 62 respiratory rate of between about 10 breaths per minute and about 30 breaths per minute. Some embodiments may define a respiratory rate of between about 16 breaths per minute and about 30 breaths per minute. The tidal volume delivered to the patient 62 from the device 10 may be between about 200 ml and about 500 ml, or between about 220 ml and about 465 ml. In some embodiments, the device 10 is configured to provide an IE ratio of between about 1.5 and 2.0. The device 10 may receive an air input at a pressure of between about 3 psi and about 5 psi. Various embodiments may be used to control ventilation or to support patient ventilation.

The process then proceeds to step 1910, which dilutes the ventilation flow from the source 66. In some embodiments, the ventilation flow may be diluted automatically, e.g., when the port 64 is integrated into the device 10 and open. Thus, in some embodiments, steps 1910 and 1908 occur simultaneously. However, in some other embodiments, dilution of the air flow may occur by opening the ports 64 (e.g., by using the movable closure 82 of FIG. 12B, which may be slidable with a finger of the medical practitioner), coupling a dilution connector 86 to the device 10 (e.g., as shown in FIGS. 15A-15C), and/or by using the nozzle switcher 94 (e.g., as shown in FIGS. 16-18C). In various embodiments, the device 10 includes at least one port 65 having an opening to an environment external of the device 10, where the device is configured so that ambient fluid enters the device through the port and mixes with the source fluid.

At step 1912, the process asks whether to remove the patient from ventilation? If the patient is not ready to be removed from ventilation, the process may return to step 1908, which continues to provide ventilation flow. However, if the medical practitioner decides to change ventilation flow parameters, the process may optionally return to step 1902, which provides a different version of the device 10 configured to provide different ventilation parameters. The process 1902-1912 may then be repeated. If the patient is ready to be removed from ventilation, the process proceeds to step 1912 and uncouples the fluidic device 10 from the patient circuit 21. The process then comes to an end.

It should be apparent that the device 10 may operate in accordance with a variety of parameters. Additionally, advantageously, various embodiments are passively operated, i.e., the device 10 switches between directing airflow to the patient 62 and the exhaust 16 without the need for a mechanical or electrical switch. However, some embodiments may include a controller and/or electronics configured to assist with the switch.

As used in this specification and the claims, the singular forms “a,” “an,” and “the” refer to plural referents unless the context clearly dictates otherwise. For example, reference to “the port” in the singular includes a plurality of ports, and reference to “the exhaust” in the singular includes one or more exhausts and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described here and shown in the figures are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

1. A fluidic device comprising:
an inlet configured to receive fluid from a pressurized source;
an outlet through which the received fluid flows towards a patient;
a device nozzle having a device nozzle axis, a device nozzle length, and a device nozzle width, wherein the inlet, the device nozzle, and the outlet share a co-planar fluid path; and
at least one port having an opening to an environment external of the device, wherein the device is configured so that ambient fluid enters the device through the port and mixes with a source fluid from the inlet to define a diluted fluid mixture, the device configured so that the diluted fluid mixture enters the device nozzle.

2. The fluidic device as defined by claim 1, wherein the inlet has a dilution nozzle defining a dilution nozzle axis.

3. The fluidic device as defined by claim 2, wherein the device nozzle axis and the dilution nozzle axis are substantially parallel.

4. The fluidic device as defined by claim 1, wherein a fluid flow path of the inlet and a fluid flow path of the outlet are parallel to a main axis of the device.

5-13. (canceled)

14. The fluidic device as defined by claim 1, wherein the inlet and the device nozzle are separately formed.

15-18. (canceled)

19. The fluidic device as defined by claim 1, further comprising:
a transition surface that is part of a patient fluid pathway leading to the outlet, the outlet being coupled to a patient;
a stepped surface that is part of a second fluid pathway leading to an exhaust;
a splitter dividing the first fluid pathway and the second fluid pathway, the splitter being asymmetrical relative to the nozzle axis.

20. The fluidic device as defined by claim 1, further comprising a fluid expansion zone distal to the inlet, configured so that fluid enters through the inlet into the fluid expansion zone that narrows towards the device nozzle, the fluid expansion zone configured to cause turbulent fluid flow.

21. A method of mechanical ventilation, the method comprising:
coupling a fluidic valve between a breathing circuit of a patient and a pressurized source, the fluidic valve having:
an inlet configured to receive fluid from a pressurized source,
a device nozzle having a device nozzle axis, a device nozzle length, and a device nozzle width; and
at least one port having an opening to an environment external of the device, wherein the device is configured so that ambient fluid enters the device through the port and mixes with a source fluid from the inlet to define a diluted fluid mixture, the device configured so that the diluted fluid mixture enters the device nozzle
a transition surface that is part of a patient fluid pathway leading to an outlet, the outlet being coupled to a patient,
a stepped surface that is part of a second fluid pathway leading to an exhaust,
a splitter dividing the first fluid pathway and the second fluid pathway, the splitter being asymmetrical relative to the nozzle axis,
wherein the inlet, the nozzle, and the outlet share a co-planar fluid path;
providing air flow into the inlet of the valve;
flowing air out of the outlet towards the patient, the air flow out of the valve producing a target PIP of between about 18 cmH20 and 30 cmH20 is achieved in the patient circuit;
flowing air out of the exhaust, after the target PIP is reached, until a PEEP of between about 6 cmH20 and 14 cmH20 is achieved in the patient circuit, wherein the steps of flowing air out of the outlet and out of the exhaust define a respiratory rate of between about 10 breaths per minute and about 30 breaths per minute.

22. The method of claim 21, wherein the fluid expansion zone makes the flow turbulent.

23. The method of claim 21, wherein the tidal volume delivered to the patient from the device is between about 200 ml and 500 ml.

24. (canceled)

25. The method of claim 21, wherein the transition surface leading to the outlet has a radius of curvature.

26-27. (canceled)

28. The method of claim 21, wherein the device is configured to that the splitter biases fluid flow towards the outlet.

29. The method of claim 21, wherein the surface leading to the exhaust is a step.

30. The method of claim 21, wherein the fluid flow channel leading to the exhaust is asymmetrical from the fluid flow channel leading to the outlet, and the exhaust fluid flow channel is shorter than the outlet fluid flow channel.

31. The method of claim 21, wherein the device is configured to provide an IE ratio of between about 1.5 and 2.0.

32. The method of claim 21, wherein the device is receiving air input at a pressure of between about 3 psi and about 5 psi.

33-37. (canceled)

38. The method of claim 21, wherein the fluidic device is configured to oscillate fluid flow between the patient outlet and the exhaust.

39. The method of claim 21, further comprising a nozzle switcher configured to transition the device between a first configuration in which the device nozzle is fluidly coupled with the port, and a second configuration in which the device nozzle is fluidly coupled with the port.

40. The device of claim 39, wherein the nozzle switcher transitions from the first configuration to the second configuration by rotating a transition feature, wherein the rotating is 45 degrees or less.

41. The device of claim 40, where an axis of rotation of the switcher is offset from the central axis of the device.

42-43. (canceled)

44. A fluidic valve for a mechanical ventilator, the valve comprising:
an inlet configured to receive fluid from a pressurized source;
a fluid expansion zone distal to the inlet, the fluid expansion zone leading to a nozzle having a nozzle axis, a nozzle length, and a nozzle width;
a transition surface that is part of a patient fluid pathway leading to an outlet, the outlet being coupled to a patient;
a stepped surface that is part of a second fluid pathway leading to an exhaust;
a splitter dividing the first fluid pathway and the second fluid pathway, the splitter being asymmetrical relative to the nozzle axis,
wherein the inlet, the nozzle, and the outlet share a co-planar fluid path.

45-53. (canceled)

54. The fluidic valve of claim 44, wherein the fluid flow channel leading to the exhaust is asymmetrical from the fluid flow channel leading to the outlet.

55-57. (canceled)

58. The fluidic valve of claim 44, wherein the inlet is substantially aligned with the device nozzle axis.

59. The fluidic device as defined by claim 1, wherein the fluid flow channel leading to the exhaust is asymmetrical from the fluid flow channel leading to the outlet and the exhaust fluid flow channel is shorter than the outlet fluid flow channel.

60. The fluidic device as defined by claim 1, wherein the fluidic device is configured to oscillate fluid flow between the patient outlet and the exhaust.
